(19) United States
(12) Patent Application Publication
(10) Pub. No.: US 2013/0253441 A1
(43) Pub. Date: Sep. 26, 2013

(54) Drug Delivery Device and Method for Sequentially Delivering at Least Two Medicaments

(71) Applicants: Carmen Patricia Keating, Aspley (AU); Malcolm Stanley Boyd, Warwickshire (GB); David Martin Leak, Lake Hopatcong, NJ (US); David Sanders, Warwick (GB); Elizabeth Anne Marshall, Newport (GB); David Aubrey Plumptre, Worcestershire (GB)

(72) Inventors: Carmen Patricia Keating, Aspley (AU); Malcolm Stanley Boyd, Warwickshire (GB); David Martin Leak, Lake Hopatcong, NJ (US); David Sanders, Warwick (GB); Elizabeth Anne Marshall, Newport (GB); David Aubrey Plumptre, Worcestershire (GB)

(73) Assignee: SanofiI-Aventis Deutschland GmbH, Frankfurt am Main (DE)

(21) Appl. No.: 13/989,361

(22) PCT Filed: Nov. 28, 2011
(86) PCT No.: PCT/EP11/71115

§ 371 Date: May 23, 2013

Related U.S. Application Data
(60) Provisional application No. 61/433,672, filed on Jan. 18, 2011.

(30) Foreign Application Priority Data
Nov. 29, 2010 (EP) 10192843.0

(51) Int. Cl. A61M 5/19; A61M 5/315

(52) U.S. Cl. CPC A61M 5/19; A61M 5/31548
USPC 604/246

(57) ABSTRACT

Various examples of a drug delivery device and corresponding method for sequentially delivering at least two medicaments via a single dispense interface are provided. In one example, the device includes a variable dose setting mechanism operably connected to a first cartridge containing a first medicament, a fixed dose setting mechanism operably connected to a second cartridge containing a second medicament, a dose setter for setting a user settable dose of the first medicament and a fixed dose of the second medicament, and a connecting feature for detachably connecting the variable dose setting mechanism to the fixed dose setting mechanism. During setting of the fixed dose of the second medicament, the rotationally driven variable dose setting mechanism and the fixed dose setting mechanism are connected via the connecting feature, however, during setting of the user settable dose of the first medicament, the rotationally driven variable dose setting mechanism and the fixed dose setting mechanism are not connected.

### CROSS REFERENCE TO RELATED APPLICATIONS

The present application is a U.S. National Phase Application pursuant to 35 U.S.C. §371 of International Application No. PCT/EP2011/071115 filed Nov. 28, 2011, which claims priority to European Patent Application No. 10192843.0 filed Nov. 29, 2010 and U.S. Provisional Patent Application No. 61/433,672 filed Jan. 18, 2011. The entire disclosure contents of these applications are herewith incorporated by reference into the present application.

### FIELD OF INVENTION

The present patent application relates to medical devices and methods of delivering at least two medicaments via a single dispense interface, where the medicaments are contained in two or more cartridges (also commonly referred to as “reservoirs”), containers or packages, each containing independent (single compound) or pre-mixed (co-formulated multiple compounds) drug agents.

### BACKGROUND

Certain disease states are preferably treated using one or more different drug agents (i.e., combination therapy). For example, in some cases it might be beneficial to treat a diabetic with a long-acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus. fjfjfididhf When combination therapy is used, certain drug agents may need to be delivered in a specific relationship to other drug agents in order to deliver the optimum therapeutic dose.

Although combination therapy may be preferred to treat certain disease states, there are a number of potential problems associated with the storage and delivery of two active drug agents. For instance, if two active drug agents are pre-mixed in a single medicament formulation they may interact with each other during long-term storage. Therefore, it is advantageous to store the active drug agents separately and only combine them at the point of delivery via injection, needle-less injection, pumps, or inhalation. However, the process for combining the two active drug agents needs to be simple and convenient for the user to perform reliably, repeatedly, and safely.

A further problem is that the quantities and/or proportions of each active drug agent making up the combination therapy may need to be varied for each user or at different stages of their therapy. For example, certain active drug agents may require a titration period to gradually introduce a patient to a “maintenance” dose. A further example is if one active drug agent requires a non-adjustable fixed dose while the other is varied in response to a patient's symptoms or physical condition. This problem means that pre-mixed medicament formulations of multiple active drug agents may not be suitable as these pre-mixed formulations would have a fixed ratio of the active drug agents, which could not be varied by the healthcare professional or user.

One known method for delivering a combination therapy is to use two separate devices each containing a cartridge with a different medicament. Accordingly, the user must take independent action with respect to the dose setters of the two devices in order to set doses of both medicaments. However, many users cannot cope with having to use more than one device and/or make the necessary accurate calculations to properly administer the required dose combination. This is especially true for users with dexterity or computational difficulties. Accordingly, there exists a strong need to provide devices and methods for the delivery of two or more drug agents that are simple for the user to perform (e.g., don't require user action with respect to multiple dose setters of multiple devices).

The disclosed device and corresponding method helps overcome the above-mentioned problems by providing separate cartridges for two or more active drug agents making up a desired combination therapy. The two or more active drug agents are only combined during delivery. Thus, the two or more active drug agents will not interact with each other during long-term storage. Additionally, the disclosed device and corresponding method is capable of achieving a wide variety of therapeutic dose profiles, therefore, making combination therapy that needs to be varied for each user or at different stages of their therapy possible. Further, the disclosed device and corresponding method allows for combination therapy without the need for users to independently set doses of multiple medicaments using different dose setters of different devices.

These and other advantages will become evident from the following more detailed description of the invention.

### SUMMARY

Disclosed herein are various examples of a drug delivery device and corresponding method for sequentially delivering (herein, sometimes referred to as “dispensing”) at least two medicaments, where each medicament contains independent (single compound) or pre-mixed (co-formulated multiple compounds) drug agents. In particular, the disclosed device and corresponding method allows a user to set and sequentially deliver (via a single dispense interface) respective doses of at least two medicaments using a single dose setter that controls at least two dose setting mechanisms, where each dose setting mechanism is associated with a different medicament.

The disclosed device and corresponding method is of particular benefit where the therapeutic response can be optimized for a specific target patient group, through control and definition of the therapeutic dose profile (i.e., the quantitative relationship between two or more medicaments (and their respective drug agents) that can be delivered to a patient). Further, the disclosed device and corresponding method is also of particular benefit where combination therapy is desirable, but not possible in a single medicament formulation for reasons such as, but not limited to, stability, compromised therapeutic performance, and toxicology.

An example advantage of sequential medicament delivery is a reduction in user force required to deliver multiple medicaments. The reduction of force is a direct consequence of maintaining a lower volumetric flow rate (multiple volumes in sequence as opposed to multiple volumes in parallel over a given time period) of medicament through the single dispense interface coupled with the fact that sequential delivery requires that only one set of mechanical losses (friction of the cartridge bung, friction or inefficiency in the delivery mechanism) is present at any given time during delivery.

The dose setter (e.g., a dial) of the device is configured to control the dose setting mechanisms (each operably coupled to a respective cartridge containing a respective medicament) such that a predefined combination of drug agents can be set via the dose setter. After the predefined combination of drug agents is set, it can be dispensed through the single dispense interface (e.g., a needle cannula). Although principally described herein as a drug delivery device capable of injecting a patient, the basic principle could be applicable to other forms of drug delivery, such as, but not limited to, inhalation, nasal, ophthalmic, oral, topical, and like devices.

By pre-defining the therapeutic relationship (i.e., the therapeutic dose profile) between various drug agents of various respective medicaments, by providing a dose setter that controls more than one dose setting mechanism, and by delivering the various medicaments via a single dispense interface, Applicants' drug delivery device helps ensure that a patient receives the optimum therapeutic combination dose without the inherent risks associated with multiple inputs and/or user error in calculating and setting the correct combination dose using multiple devices and/or multiple dose setters. Accordingly, the disclosed device and corresponding method is of particular benefit to users with dexterity or computational difficulties in terms of both improved compliance with a prescribed therapy and patient safety.

One or more of the medicaments making up the combination dose may be a fluid, defined herein as a liquid, gas or powder that is capable of flowing and that changes shape at a steady rate when acted upon by a force tending to change its shape. One or more of the medicaments may be a solid, powder, suspension of slurry that may be carried, solubilized or otherwise dispensed with another fluid medicament. In one example, the therapeutic combination dose comprises a first and a second medicament contained in respective cartridges. Both medicaments may be fluids or one medicament may be a fluid and the other may be a powder that is either dissolved or entrained in the fluid medicament before it is delivered via the a single dispense interface.

Possible drug combinations may include insulin, insulin analogs or insulin derivatives, and GLP-1 or GLP-1 analogs, however, other drugs or drug combinations, such as an analgesics, hormones, beta agonists or corticosteroids, or a combination of any of the above-mentioned drugs could be used with Applicants' proposed device and method.

As used herein, the term “insulin” shall mean insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

In one example of the drug delivery device, the drug delivery device includes a dose setter for setting a user settable dose of a first medicament and a fixed dose (i.e., non-user settable dose) of a second medicament, a dose button, a single dispense interface, a first cartridge containing multiple doses of the first medicament, and a second cartridge containing multiple doses of the second medicament. The dose button can be any type of mechanism that triggers the delivery procedure, whether driven mechanically or through a combination of electronics and mechanics. The button can physically move or be a touch sensitive virtual button, for example, a touch sensitive screen.

As used herein, a “user settable dose” means a dose that the user (e.g., a patient or health care provider) can choose and physically manipulate the device to set. In other examples, a user settable dose can be set remotely through the use of wireless communication (Bluetooth, WiFi, satellite, etc.) or the dose could be set by another integrated device, such as a blood glucose monitor after performing a therapeutic treatment algorithm. By “fixed dose,” it is meant that a user (or any other input) cannot set a desired dose, rather, the user can only set a predetermined dose that is defined by the fixed dose setting mechanism.

The single dispense interface is configured for fluid communication with the first and second cartridges. The dispense interface can be any type of outlet that allows the two or more medicaments to exit the device and be delivered to the patient. Types of interfaces include needle cannulas, catheters, atomizers, pneumatic injectors, needle-less injectors, mouthpieces, nasal-applicators, and the like. The combination of medicaments may be delivered via the single dispense interface as discrete units or as a mixed unit, thus providing a combination therapy that, from a user's perspective, is achieved in a manner that very closely matches the currently available injection devices that use standard needles.

In another example, the drug delivery device includes a rotationally driven variable dose setting mechanism operably connected to a first cartridge containing a first medicament, a fixed dose setting mechanism operably connected to a second cartridge containing a second medicament, a dose setter for setting a user settable dose of the first medicament and a fixed dose of the second medicament, and a connecting feature for detachably connecting the rotationally driven variable dose setting mechanism to the fixed dose setting mechanism. During setting of the fixed dose of the second medicament, the rotationally driven variable dose setting mechanism and the fixed dose setting mechanism are connected via the connecting feature, however, during setting of the user settable dose of the first medicament, the rotationally driven variable dose setting mechanism and the fixed dose setting mechanism are not connected via the connecting feature.

The dose setter may include the connecting feature and may be axially slidably linked (via at least one splined (i.e., axial) groove in the dose setter) to the rotationally driven variable dose setting mechanism while being detachably linked (via the connecting feature) to the fixed dose setting mechanism. When the dose setter is linked to the fixed dose setting mechanism via the connecting feature, the dose setter may be axially displaced in a proximal direction to set the fixed dose of the second medicament, and when the dose setter is detached from the fixed dose setting mechanism, the dose setter can be rotated to set the user settable dose of the first medicament. Alternatively, the dose setter may be configured to set both the fixed dose of the second medicament and the user settable dose of the first medicament by merely rotating the dose setter. Such a dose setter may be part of the rotationally driven variable dose setting mechanism.

The dose setter may be configured to set the fixed dose of the second medicament before the user settable dose of the first medicament and to deliver the user settable dose of the first medicament before the fixed dose of the second medicament. After the fixed dose of the second medicament is set, the dose setter may detach from the fixed dose setting mechanism. After (i) the user settable dose of the first medicament and the fixed dose of the second medicament are set and (ii) the user settable dose of the first medicament is delivered, the dose setter may re-attach to the fixed dose setting mechanism via the connecting feature such that axial displacement of the dose setter (whether caused by rotation of the dose setter along a helical path or axial translation) in the distal direction causes the fixed dose of the second medicament to be delivered.

The drug delivery device disclosed herein may be designed in such a way as to limit its use to exclusive first and second cartridges through employment of dedicated or coded features.

Applicants' present disclosure also covers a method of setting and delivering a variable dose of a first medicament and a fixed dose of a second medicament from separate cartridges of a drug delivery device. In one example, the method uses a device as described above and involves the steps of first setting a dose of the second medicament followed by a dose of the first medicament using a dose setter. Next, the device is activated and the user settable dose of the first medicament is delivered followed by the fixed dose of the second medicament. Both doses are delivered via a single dispense interface. To set the fixed dose of the second medicament, the dose setter may be pulled in the proximal direction. After the fixed dose of the second medicament is set, the user settable dose of the first medicament may be set by merely rotating the dose setter. Alternatively, the doses of the first and second medicaments may both be set by rotating the dose setter.

In another example, the method involves (i) attaching a single dispense interface to the distal end of a drug delivery device such that the proximal end of the single dispense interface is in fluidic communication with both a first medicament and a second medicament, (ii) using a dose setter to set both a user settable dose of the first medicament and a fixed dose of the second medicament, (iii) inserting the distal end of the single dispense interface into the patient at the desired administration site, and (iv) dispensing the fixed dose of the second medicament followed by the user settable dose of the first medicament.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Various examples of Applicants' drug delivery device and corresponding method are described herein with reference to the following drawings, wherein like numerals denote like entities:

FIG. 1 illustrates an example therapeutic dose profile that can be achieved with the drug delivery device of Applicants' disclosure;

FIG. 2 illustrates an example of the drug delivery device;

FIG. 3 illustrates another example of the drug delivery device;

FIG. 4 illustrates some of the internal structure of the variable dose setting mechanism of the drug delivery device shown in FIG. 3;

FIG. 5 illustrates the internal structure of the variable dose setting mechanism shown in FIG. 3;

FIG. 6a illustrates the drug delivery device shown in FIG. 3 at the beginning of dose setting of a fixed dose of the second medicament;

FIG. 6b illustrates the drug delivery device shown in FIG. 3 during dose setting of a fixed dose of the second medicament;

FIG. 6c illustrates the drug delivery device shown in FIG. 3 during dose setting of a user settable dose of the first medicament;

FIG. 6d illustrates the drug delivery device shown in FIG. 3 during dose delivery of the user settable dose of the first medicament; and

FIG. 6e illustrates the drug delivery device shown in FIG. 3 during dose delivery of the fixed dose of the second medicament.

### DETAILED DESCRIPTION

The drug delivery device disclosed herein is capable of sequentially delivering at least two medicaments through a single dispense interface.

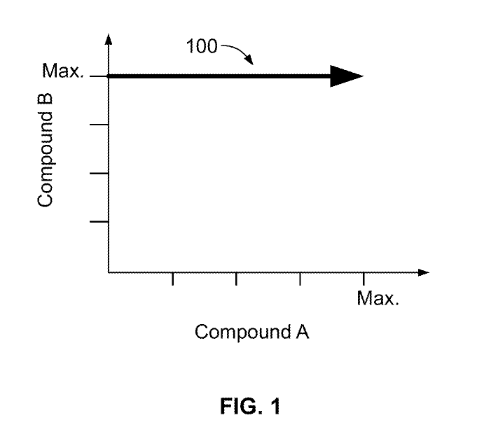

In one example, the drug delivery device includes a dose setter for setting both a user settable (i.e., variable) dose of a first medicament and a fixed dose of a second medicament. After both doses have been set, the user activates the drug delivery device and the user settable dose of the first medicament is delivered followed by the fixed dose of the second medicament. Herein, “activating” the drug delivery device may comprise a single action or multiple actions. The therapeutic dose profile 100 representing the relationship between the user settable dose of the first medicament and the fixed dose of the second medicament is illustrated in FIG. 1, where Compound A represents the first medicament and Compound B represents the second medicament. As shown, the user settable dose of the first medicament may vary while the dose of the second medicament remains fixed, which may be beneficial for certain therapies. Profiles of this type are not achievable with a device having a single cartridge that contains a co-formulated combination dose where the concentration of the various constituent parts is constant (x mg/ml).

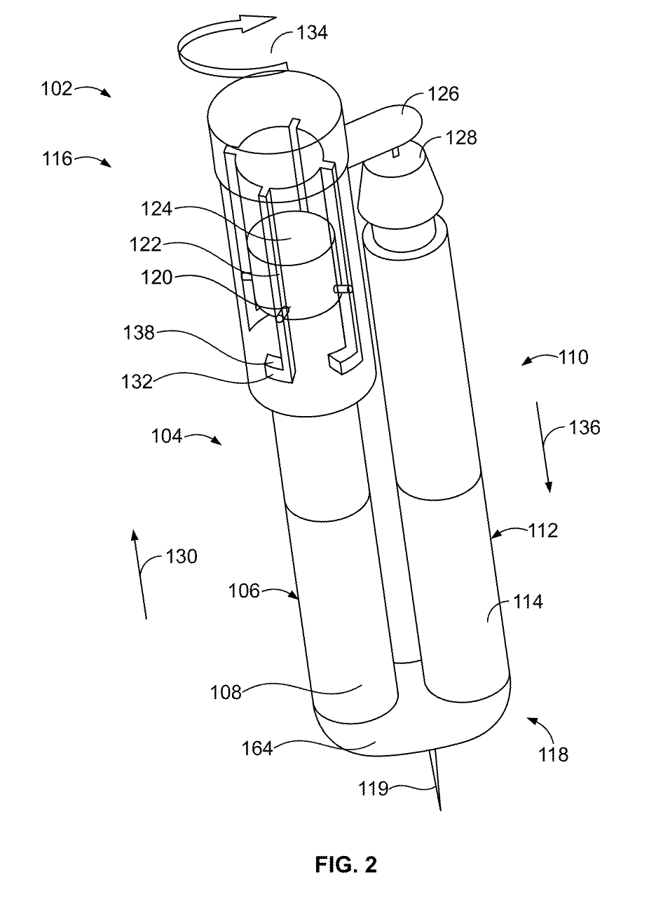

FIG. 2 illustrates an example of the drug delivery device 102. As shown, the drug delivery device 102 includes a rotationally driven variable dose setting mechanism 104 (a “variable dose setting mechanism”) operably connected to a first cartridge 106 containing a first medicament 108, a fixed dose setting mechanism 110 operably connected to a second cartridge 112 containing a second medicament 114, a splined dose setter 116, and a needle assembly 118 having a single dispense interface 119.

The splined dose setter 116 is operably connected to both dose setting mechanisms 104, 110 such that a user can set both a user settable dose of the first medicament 108 and a fixed dose of the second medicament 114 via the splined dose setter 116. The splined dose setter 116 is operably connected to the variable dose setting mechanism 104 via engagement of the four radial protrusions 120 of the variable dose setting mechanism and the four splined (i.e., axial) grooves 122 located on an inner surface of the dose setter 116. As shown, the four radial protrusions 120 are connected to the dose setter 124 of the variable dose setting mechanism 104. Accordingly, rotating the splined dose setter 116 of the device 102 causes corresponding rotation of the dose setter 124 of the variable dose setting mechanism 104, which sets a dose of the first medicament 108. Other examples of the device 102 may include any number of radial protrusions 120 and corresponding axial grooves 122. The radial protrusions 120 may be separate parts that are connected to the dose setter 124 of the variable dose setting mechanism 104, perhaps by pins or screws, or they may be part of the dose setter 124 itself.

The splined dose setter 116 is operably detachably connected to the fixed dose setting mechanism 110 via the splined dose setter's connecting feature 126. The connecting feature 126 engages the proximal end 128 of the fixed dose setting mechanism 110 (i.e., the dose setter of the fixed dose setting mechanism) via a 2-way clip, bump or snap connection mechanism (not shown). When the splined dose setter 116 is connected to the dose setter 128 of the fixed dose setting mechanism 110 (via the connecting feature 126), proximal axial movement 130 of the splined dose setter 116 causes corresponding axial movement of the dose setter 128 of the fixed dose setting mechanism 110, which sets a dose of the second medicament 114.

Once the fixed dose of the second medicament has been set, stop features in the fixed dose setting mechanism (not shown, but such as those known in the art) prevent further proximal axial movement of dose setter 128. At this point the detachability of the connecting feature 126 allows the variable dose setting mechanism 104 to transition from being connected to the fixed dose setting mechanism 110 (via the connecting feature 126 of the splined dose setter 116) during dose setting of the second medicament 114 to being disconnected from the fixed dose setting mechanism 110 during dose setting of the first medicament 108. During medicament delivery, the detachability of the connecting feature 126 allows the variable dose setting mechanism 104 to transition from being disconnected from the fixed dose setting mechanism 110 during delivery of the user settable dose of the first medicament 108 to being connected to the fixed dose setting mechanism 110 (via the connecting feature 126 of the splined dose setter 116) upon full delivery of the fixed dose of the second medicament 114.

To further illustrate the various features of the above-described example of the drug delivery device 102, an example method using the device 102 will now be described. First, a user sets a fixed dose of the second medicament 114. To set the fixed dose, the user pulls the splined dose setter 116 of the device 102 upwards (i.e., in the proximal direction 130). Because the connecting feature 126 is engaged with the dose setter 128 of the fixed dose setting mechanism 110, as the splined dose setter 116 is pulled upwards, the dose setter 128 of the fixed dose setting mechanism 110 is forced in the proximal direction 130 as well, thereby setting a fixed dose of the second medicament 114. In order to accomplish this result, the force required to disengage the connecting feature 126 from the fixed dose setting mechanism 110 would preferably be a safe margin greater than the force required to set the fixed dose of the second medicament 114.

While the connecting feature 126 is engaged with the fixed dose setting mechanism 110 it is not possible for the user to rotate the splined dose setter 116 and therefore not possible to set a dose of the first medicament 108. Rather, the dose setter 116 can only slide in the axial direction. Engagement of the radial protrusions 120 with the splined grooves 122 in the splined dose setter 116 help guide the axial movement of the splined dose setter 116 during dose setting of the second medicament 114. The axial distance traveled by the splined dose setter 116 during dose setting of the second medicament 114 has no effect on the variable dose setting mechanism 104.

After the fixed dose of the second medicament 114 is set, the user sets a user settable dose of the first medicament 108. When the fixed dose of the second medicament 114 is fully set, the dose setter 128 of the fixed dose setting mechanism 110 cannot be further displaced in the proximal direction 130 (i.e., the dose setter 128 of the fixed dose setting mechanism 110 is fully extended). Consequently, as the user continues to pull the splined dose setter 116 of the device 102 in the proximal direction 130 (to a predefined axial position) the connecting feature 126 disengages from the dose setter 128 of the fixed dose setting mechanism 110. As noted above, the connecting feature 126 is designed such that its disengagement force is a safe margin greater than that required to set a dose of the second medicament 114, but not so high that it is outside user capabilities. This ensures that the user can disengage the connecting feature 126 from the fixed dose setting mechanism 110 and also that disengagement does not occur until after the fixed dose of the second medicament 114 is fully set.

Disengagement of the connecting feature 126 from the fixed dose setting mechanism 110 allows the user to rotate the splined dose setter 116 and thus to rotate the dose setter 124 of the variable dose setting mechanism 104 in order to set a desired dose of the first medicament 108. Rotating the splined dose setter 116 causes corresponding rotation of the dose setter 124 of the variable dose setting mechanism 104. Thus, the user can rotate the splined dose setter 116 until the desired user settable dose of the first medicament 108 is set. To eliminate axial movement of the splined dose setter 116 during dose setting of the first medicament, the distal ends of the splined grooves 122 may include cutouts 132 in which the radial protrusions 120 preferably enter upon initial rotation of the splined dose setter 116. In order to facilitate this, the axial length of the splined grooves 122 and the relative axial position of the cutouts 132 with respect to the connecting feature 126 are such that after the fixed dose of the second medicament 114 is set and the connecting feature 126 disengages from the fixed dose setting mechanism 110, the radial protrusions 120 enter the cutouts 132 upon counter-clockwise rotation 134 of the splined dose setter 116.

Once the first medicament 108 is set to the desired user settable dose, the splined dose setter 116 of the device 102 can be pushed downwards (in the distal direction 136) to deliver the set dose of the first medicament 108. When the user pushes down on the splined dose setter 116, the protrusions 120 are forced down by the proximal surfaces 138 of the cutouts 132, which activates the variable dose setting mechanism 104 by actuating the dose setter 124 of the variable dose setting mechanism 104.

After the user settable dose of the first medicament 108 has been delivered and the dose setters 124, 116 of the variable dose setting mechanism 104 and the device 102 have retuned to their pre-first-medicament-setting position, the connecting feature 126 is realigned with the fixed dose setting mechanism 110. Thus, by pushing the splined dose setter 116 in the distal direction 136 (i.e., further activating the device 102), the connecting feature 126 re-attaches to the dose setter 128 of the fixed dose setting mechanism 110 and continued pushing in the distal direction 136 causes delivery of the fixed dose of the second medicament 114. After the fixed dose of the second medicament 114 is delivered, the device 102 is ready for setting of the next doses.

In another example (not shown) of the drug delivery device 102, the dose setter 116 of the device 102 has a helical groove (e.g., threads) instead of splined (i.e., axial) grooves 122 and the dose setter 124 of the variable dose setting mechanism 104 has a compatible helical protrusion (e.g., threads) instead of radial protrusions 120. The helical groove and corresponding protrusion are configured such that after a fixed dose of the second medicament 114 is set and the connecting feature 126 has disengaged from the fixed dose setting mechanism 110, the dose setter 116 cannot move further in the proximal direction 130 without rotating the dose setter 124 of the variable dose setting mechanism 104 (i.e., the distal end of the helical groove of the dose setter has been reached and further rotation causes the dose setter 124 of the variable dose setting mechanism 104 to rotate).

In this example, to set a fixed dose of the second medicament 114, the user rotates the dose setter 116 of the device 102 instead of pulling it upwards 130. Thus, the connecting feature 126 in this example is preferably a separate part that interfaces with the dose setter 116 of the device 102 in such a way that it remains rotationally fixed as the user rotates the dose setter 116. This may be accomplished if the portion of the connecting feature 126 that interfaces with the dose setter 116 is not fixed to the dose setter 116, thus allowing the dose setter 116 to rotate relative to the connecting feature 126. However, axial movement of the dose setter 116 should cause corresponding axial movement of the connecting feature 126 in order to set the fixed dose of the second medicament 114. This example only requires the user to perform one action (i.e., rotation) to set respective doses of both the first and second medicaments 108, 114. The user simply rotates the dose setter 116 until a fixed dose of the second medicament 114 is set and then continues to rotate the dose setter 116 until a user settable dose of the first medicament 108 is set.

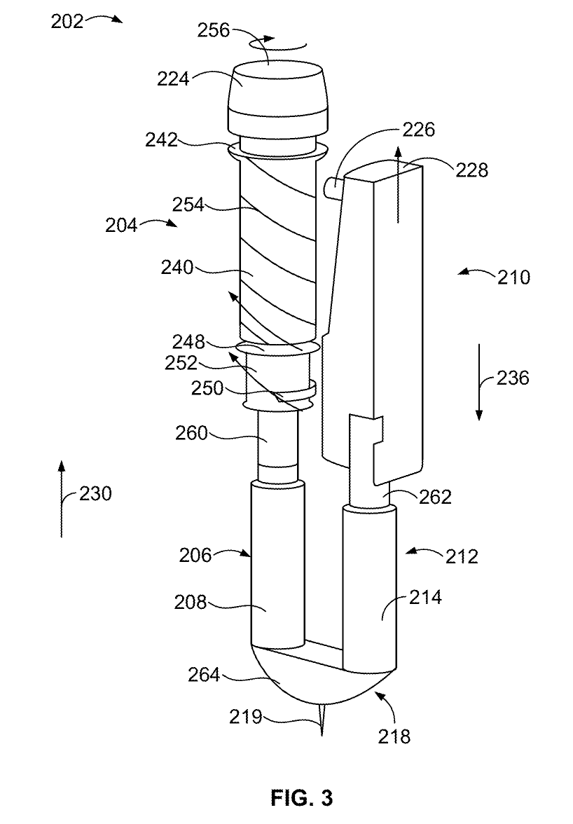

FIG. 3 shows another example of the drug delivery device 202. As shown, like the examples described above, the drug delivery device 202 includes a rotationally driven variable dose setting mechanism 204 operably connected to a first cartridge 206 containing a first medicament 208, a fixed dose setting mechanism 210 operably connected to a second cartridge 212 containing a second medicament 214, a connecting feature 226, and a needle assembly 218 having a single dispense interface 219. However, unlike the examples described above, the device 202 utilizes the dose setter 224 (i.e., dial) of the variable dose setting mechanism 204 to set the user-settable dose of the first medicament 208 and the non user-settable dose of the second medicament 214. This eliminates the need for a splined dose setter 216 and only requires the user to perform one action (i.e., rotation) to set respective doses of both medicaments 208, 214.

In this example, the connecting feature 226 is part of, or operably connected to, the fixed dose setting mechanism 210 and is detachably connected (shown detached in FIG. 3) to the dial sleeve 240 of the variable dose setting mechanism 204 via the lifting collar 242. The connecting feature 226 and lifting collar 242 are configured such that the variable dose setting mechanism 204 can transition from being connected to the fixed dose setting mechanism 210 (via engagement of the connecting feature 226 and collar 242) during dose setting of the second medicament 214 to being disconnected from the fixed dose setting mechanism 210 during dose setting of the first medicament 208. When the variable dose setting mechanism 204 is connected to the fixed dose setting mechanism 210, proximal axial movement 230 of the dial sleeve 240 of the variable dose setting mechanism 204 causes corresponding axial movement of the dose setter 228 of the fixed dose setting mechanism 210, thus setting a fixed dose of the second medicament 214. During medicament delivery, the connecting feature 226 and lifting collar 242 allow the variable dose setting mechanism 204 to transition from being disconnected from the fixed dose setting mechanism 210 during delivery of the first medicament 208 to being connected to the fixed dose setting mechanism 210 (via engagement of the connecting feature 226 and collar 242) during delivery of the second medicament 214.

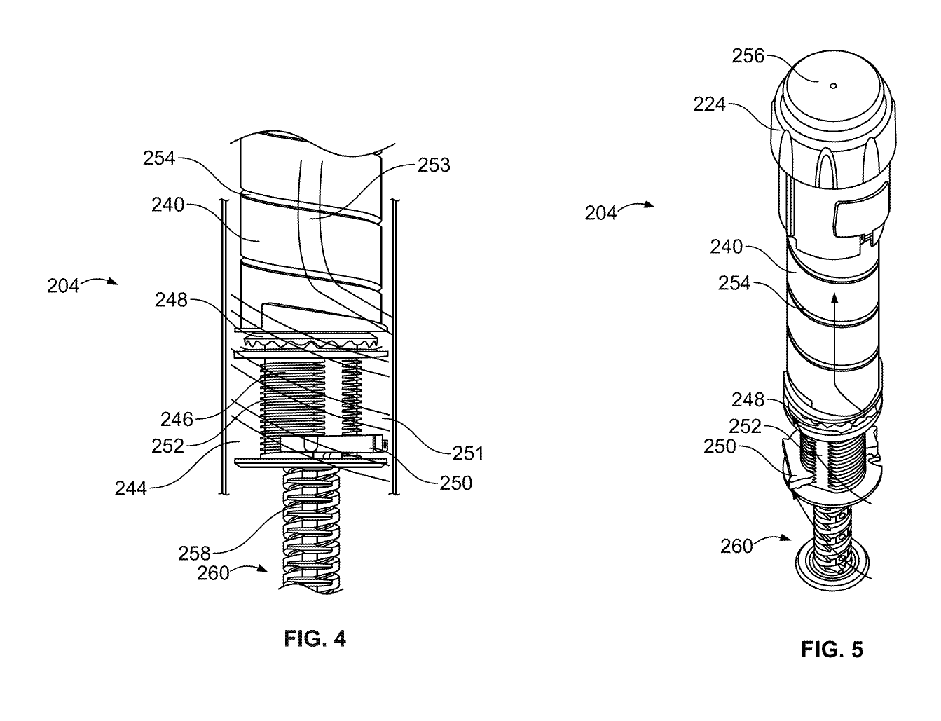

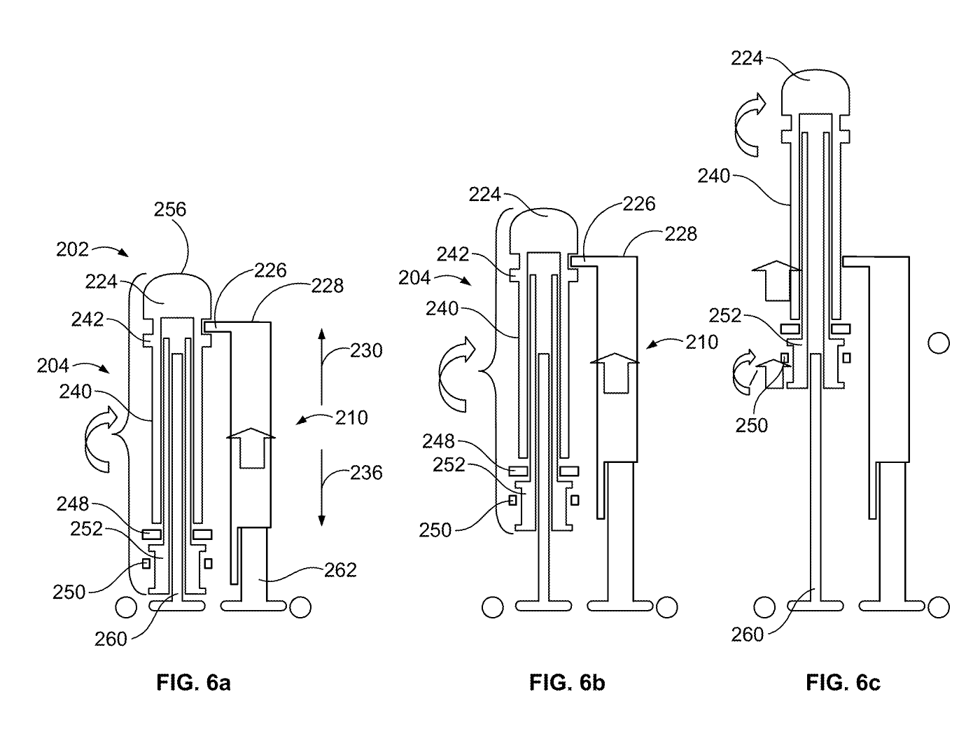

FIGS. 4 and 5 illustrate example features of the internal structure of the variable dose setting mechanism 204 that enable sequential delivery of the first and second medicaments 208, 214. As shown in FIG. 4, the body/housing 244 includes one or more continuous grooves 246 that are helical near the distal end and transitions to being generally vertical/axial. During operation (i.e., during rotation of the dose setter 224 of the variable dose setting mechanism 204 to set doses of both the first and second medicaments 208, 214), the clicker 248 and the 300 unit counter 250 (the “counter”) are initially located in the helical portions 251 of the body/housing grooves 246. As the dose setter 224 is rotated, the clicker 248 and the counter 250 follow the helical path of the body/housing grooves 246 at which time a fixed dose of the second medicament 214 is being set. Then, at a first predetermined axial position that corresponds to the fixed dose of the second medicament 214 being fully set, the clicker 248 enters the vertical portion 253 of the body/housing grooves 246, however, the counter 250 is still located in the helical portion 251 of the grooves 246. At a second axial position, the counter 250 enters the vertical portion 253 of the body/housing grooves 246. When the clicker 248 is in the vertical portion 253 of the grooves 246, the user settable dose of the first medicament 208 is being set.

The counter 250 is configured to count the cumulative number of variable doses set over the life of the variable dose setting mechanism 204 and limits the cumulative total to a maximum dose limit (for example the available volume in the cartridge), whereas the clicker 248 counts the amount of the variable dose set during a single setting action of the device 204. Variable dose counting (using the clicker 248) for a single setting action occurs when the clicker 248 is engaged with the vertical portion 253 of the body/housing grooves 246. The cumulative number of variable doses counted (using the counter 250) starts when both the counter 250 and clicker 248 are engaged with the vertical portion 253 of the body/housing grooves 246 during dose setting but also when the clicker 248 is in the vertical portion 253 of the grooves 246 and the counter 250 is in the helical portion 251 of the grooves 246 during delivery. The body/housing grooves 246 are configured so that when the 300 unit stop (not shown) is reached, the variable dose setting mechanism 204 has dialed a total of 300 units. At this point, during delivery, the counter 250 enters the helical portion 251 at the same time as the clicker 248, so that no further counting occurs during delivery and the variable dose setting mechanism 204 remains locked after delivery so that further dose setting is not possible.

In functional terms, initial rotation of the dose setter 224 of the variable dose setting mechanism 204 causes rotation of the dial sleeve 240 and drive sleeve 252 (including the clicker 248 and the counter 250) around a prescribed helical path defined by a groove 254 in the dial sleeve 240. The helical path of the dial sleeve groove 254 matches that of the internal helical groove 246 in the body/housing 244 of the variable dose setting mechanism 204 into which the clicker 248 and the counter 250 engage. As these helical paths are identical, the clicker 248 does not incur any relative movement with respect to the drive sleeve 252 and therefore does not begin counting. In addition, while the counter 250 rotates in the helical portion 251 of the body/housing groove 246, it does not incur any relative movement with respect to the drive sleeve 252 and thus does not count any doses towards the cumulative total.

The axial component of this helical (and un-counted) motion is used to lift the dose setter 228 of the fixed dose setting mechanism 210 to its fully set point. After the fixed dose of the second medicament 214 is set, the lifting collar 242 disconnects from the connecting feature 226 and the clicker 248 enters the vertical portion 253 of the body/housing grooves 246. Further rotation of the dose setter 224 of the variable dose setting mechanism 204 sets a variable dose of the first medicament 208. This rotation causes the dial and drive sleeves 240, 252 to rotate relative to the clicker 248, thus resulting in the clicker 248 counting the variable dose. During dose setting of the first medicament 208, the clicker 248 is constrained by the vertical portion 253 of the body/housing grooves 246 such that it only moves axially. At some point the counter 250 enters the vertical portion 253 of the body/housing groove 246 and begins counting doses towards the cumulative total (as it is constrained to move axially, whereas the drive sleeve 252 continues to follow the helical path defined by the groove 254 in the dial sleeve 240).

After the fixed dose of the second medicament 214 and the user settable dose of the first medicament 208 are set, activation of the device 102 by actuating the dose button 256 disengages the dial sleeve 240 from the drive sleeve 252 rotationally, and causes delivery of the first medicament dose followed by the second medicament dose. During delivery, the dial sleeve 240 follows its helical path back in the distal direction 236, however, the drive sleeve 252, now de-coupled from the dial sleeve 240 through actuation of the clutch (not shown), but fixed in rotation by engagement of the clicker 248 in the vertical portion 253 of the body/housing grooves 246, moves axially without rotation, thus causing the first medicament dose to be delivered as the lead screw 260 forces the bung (not shown) of the first cartridge 206 in the distal direction 236. After the user settable dose of the first medicament 208 is delivered, the lifting collar 242 re-engages with the connecting feature 226 and as the dial sleeve 240 continues to move in the distal direction 236 along its helical path it causes the fixed dose setting mechanism 210 to deliver the fixed dose of the second medicament 214. The dial sleeve follows an accurately defined path relative to the housing and will therefore reengage the connecting feature 226 as it rotates back distally.

When the lifting collar 242 re-engages with the connecting feature 226, the clicker 248 enters the helical portion of the body/housing groove 246, thus allowing the variable dose setting mechanism 204 to rotate back down the helical portion 251 of the body/housing grooves 246. The first medicament 208 is not delivered as the variable dose setting mechanism 204 rotates back down the helical portion 251 of the body/housing grooves 246 since the pitch of the helical portion 251 of the body/housing groove 246 is designed to match the pitch of the helical thread 258 between drive sleeve 252 and lead screw 260. During delivery, the counter 250 initially moves axially along the vertical portion 253 of the body/housing grooves 246, after which it enters the helical portion 251 of the body/housing grooves 246. When the counter 250 is located in the helical portion 251 of the body/housing grooves 246 but he clicker 248 is still located in the vertical portion 253 of the grooves 246, the counter 250 counts doses towards the cumulative total as it rotates and the drive sleeve 240 travels axially.

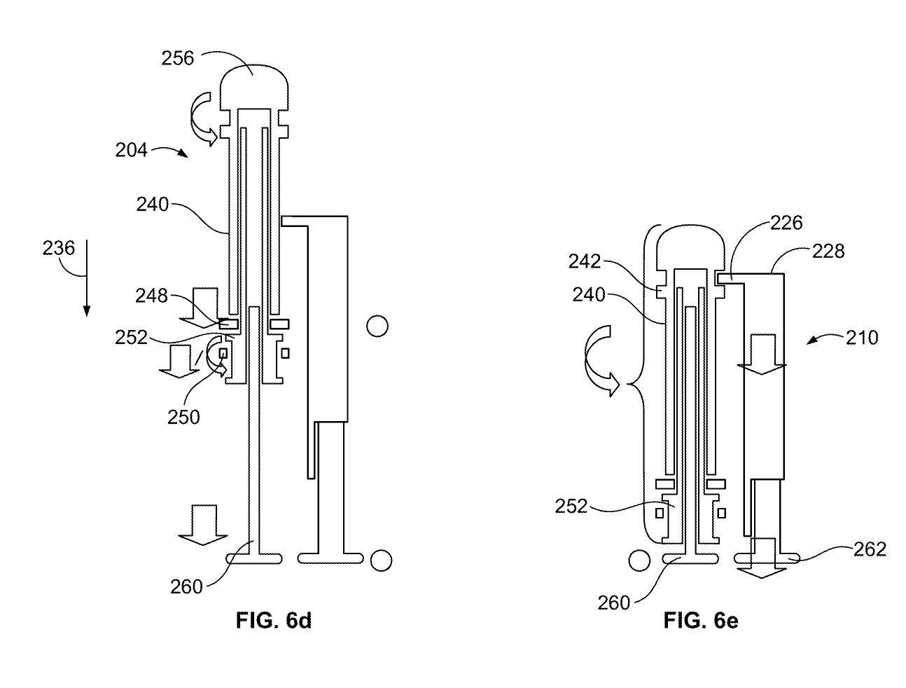

To further illustrate the various features of the above-described example of the drug delivery device 202, an example method using the device 202 will now be described with reference to FIGS. 6a-6e. In these figures, the arrows indicate the direction of motion and the circles indicate components that are fixed. First, the user sets a fixed dose of the second medicament 214. As shown in FIGS. 6a and 6b, initial rotation of the dose setter 224 of the variable dose setting mechanism 204 causes the dial and drive sleeves 240, 252 (which are in clutched engagement) to rotate together up the helical path defined by the groove 254 in the dial sleeve 240, thereby lifting the dose setter 228 of the fixed dose mechanism 210 to its set point via engagement of the lifting collar 242 and the connecting feature 226. The counter 250 and the clicker 248 follow the helical path defined by the grooves 246 in the body/housing 244, which is identical to the helical path defined by the groove 254 in the dial sleeve 240, and thus do not count the dialed dose. Upon reaching its set point, the fixed dose setting mechanism 210 disengages from the variable dose setting mechanism 204. The fixed dose piston rod 262 remains fixed during this phase through the operation of a one way ratchet (not shown).

Upon reaching the fixed dose set point, the clicker 248 engages the vertical portion 253 of the body/housing grooves 246, throughout which the variable dose is counted. Continued rotation of the dose setter 224 and corresponding rotation of the dial sleeve 240 forces the drive sleeve 252 to rotate relatively to the lead screw 260 (see FIG. 6c), which is held in place by a nut (not shown). During this phase, the counter 250 initially rotates relative to the body/housing and then, at some point, enters the vertical portion 253 of the body/housing grooves 246 and begins counting cumulative variable doses.

Turning to FIG. 6d, next, the user delivers the user settable dose of the first medicament 208 by activating the device 102 via actuation of the dose button 256. During delivery of the user settable dose of the first medicament 208, the dial sleeve 240 is rotationally de-coupled from the drive sleeve 252. This action allows the dial sleeve 240 to rotate helically back into the body 244 of the device 204, and the drive sleeve 252 to move axially thus causing the lead screw 260 to move in the distal direction 236 thereby delivering the first medicament dose by forcing the bung (not shown) of the first cartridge 206 in the distal direction 236. Both the clicker 248 and the drive sleeve 240 move axially during this phase. The counter 250 initially moves axially along the vertical portion 253 of the body/housing grooves 246 after which it enters the helical portion 251 of the body/housing grooves 246 during which it counts doses as it rotates relative to the axially moving drive sleeve 252. Eventually, the clicker 248 enters the helical portion 251 at which point the clicker 248 and counter 250 rotate together with the drive sleeve and the counter 250 stops counting doses. Note that when the 300 unit or other suitable unit stop (not shown) has been reached (a total of 300 units have been dialed) the counter 250 prevents further dialing. When this last dose is delivered, both the clicker 248 and counter 250 are designed to enter their respective helical grooves together so that in this final dose no counting occurs during delivery and at the end of the dose the device 204 is locked out against further dialing.

Turning to FIG. 6e, towards the end of the variable delivery phase, the lifting collar 242 re-engages with the connecting feature 226. After re-engagement, delivery of the fixed dose of the second medicament 214 begins. As the dial sleeve 240 continues to move in the distal direction 236, the lifting collar 242, which is engaged with the dose setter/dispenser 228 (via the connecting feature 226) of the fixed dose setting mechanism 210, forces the dose setter/dispenser 228 in the distal direction 236 thus forcing the piston 262 in the distal direction 236 through the operation of the ratchet. This distal movement 236 of the piston 262 causes the fixed dose of the second medicament 214 to be delivered as it forces the bung (not shown) of the second cartridge 212 in the distal direction 236. The variable dose setting mechanism 204 does not dispense during this phase as the drive sleeve 252 and clicker 248 rotate around a helical path that matches that of the lead screw 260.

In any of the above-described examples of the device 102, 202 the connecting feature 126, 226 may be configured to give an audible and/or tactile feedback to the user when the end of dose delivery has been reached. Similarly, disengagement of the connecting feature 126, 226 after setting a fixed dose of the second medicament 114, 214 may provide an audible and/or tactile feedback to the user, thus apprising the user that the fixed dose of the second medicament 114, 214 is set and that the user may now set the desired user settable dose of the first medicament 108, 208.

The drug delivery device disclosed herein is most suitable to be a modular disposable or re-usable device in terms of managing medicament wastage because there is a reasonable probability that one of the medicaments will be exhausted before the other unless there is a strict 1:1 ratio between the delivered doses of the two medicaments. Where each drive mechanism is resettable, new cartridges can be inserted and the device can continue to be used. Likely embodiments for a modular disposable device could be, but are not limited to, replacement of the entire device fitted with new cartridges and replacement of the drive mechanisms fitted with new cartridges. Regardless of whether the device is modular disposable or re-usable, suitable re-engagement features may be integrated into the device to facilitate the alignment and fastening of the individual device components together in a robust, intuitive and user-friendly fashion. Such modular disposable arrangements may preferably be configured to render the individual elements inoperable until they are correctly connected together.

A re-usable device may feature spindles, lead screws and/or pistons that can be back wound into their respective drive mechanisms once they reach their respective limits of travel. This may be achieved by placing the device into a reset state, for example by removing one or both of the cartridges, after which the respective body nut(s) holding the spindles or pistons becomes free to rotate relative to the device body. Manual rotation of a body nut would then cause the respective spindle or piston to be rotated and this in turn will cause the spindle or piston to wind its way back up into the device body and return to its initial position. In addition to this functionality, the re-usable device may have a mechanism for easy replacement of the cartridges after resetting their respective spindles or pistons.

In all of the examples described above, the first and second medicaments are capable of being delivered via a single dispense interface 119, 219. To attach a single dispense interface to the first and second cartridges, the first and second cartridges may have respective attachment means at their distal end. Alternatively, the cartridges may be housed in respective cartridge holders and the cartridge holders may have respective attachment means.

The attachment means may be compatible with a needle assembly 118, 218 that includes a hub 164, 264 and a single dispense interface 119, 219. The needle assembly 118, 218 may be removable and may be either disposable or reusable. Such a needle assembly is shown in FIGS. 2 and 3. The needle assembly 118, 218 can take any form, provided that it allows for fluid communication between the first and second medicaments and the single dispense interface 119, 219. An exemplary needle assembly 118, 218 may include what is referred to in the art as a “2-to-1 needle” configuration.

Although not shown, the needle assembly may be supplied by a manufacturer in a protective and sterile capsule or container that completely or partially contains the needle assembly. A user may peel and/or rip open a seal or the container itself to gain access to the sterile single dispense interface. In some instances it might be desirable to provide two or more seals for each end of the needle assembly. The seal may allow for the display of information required by regulatory labeling requirements.

Attachment of the needle assembly to the drug delivery device via the hub creates a fluid connection between dispense interface and the first and second medicaments.

Although the various examples of the drug delivery device described herein comprise a single dispense interface, other examples may comprise multiple dispense interfaces, for example, a different dispense interface for each respective cartridge/medicament. Multiple dispense interfaces may be advantageous when simultaneous injection of 2 or more medicaments is desirable, but where co-injection of a particular combination of these medicaments into the same injection site may adversely effect the Pharmaco-kinetic (PK) profile of one or more or the medicaments. By way of an example, it is understood that dilution of certain long-acting basal insulins may alter their PK profile in-vivo.

Examples of the drug delivery device and corresponding method have been described. Those skilled in the art will understand, however, that changes and modifications may be made to these examples without departing from the true scope and spirit of the present invention, which is defined by the claims.

1. A drug delivery device for sequentially delivering at least two medicaments, the drug delivery device comprising:
a rotationally driven variable dose setting mechanism operably connected to a first cartridge containing a first medicament;
a fixed dose setting mechanism operably connected to a second cartridge containing a second medicament;
a dose setter for setting a user settable dose of the first medicament and a fixed dose of the second medicament; and
a connecting feature for detachably connecting the rotationally driven variable dose setting mechanism to the fixed dose setting mechanism, wherein during setting of the fixed dose of the second medicament, the rotationally driven variable dose setting mechanism and the fixed dose setting mechanism are connected via the connecting feature, and wherein during setting of the user settable dose of the first medicament, the rotationally driven variable dose setting mechanism and the fixed dose setting mechanism are not connected.

2. The drug delivery device of claim 1, wherein the dose setter includes at least one splined groove.

3. The drug delivery device of claim 1, wherein the dose setter includes the connecting feature.

4. The drug delivery device of claim 3, wherein the dose setter is axially slidably linked to the rotationally driven variable dose setting mechanism and detachably linked to the fixed dose setting mechanism via the connecting feature.

5. The drug delivery device of claim 1, wherein, when the dose setter is linked to the fixed dose setting mechanism via the connecting feature, the dose setter can be axially displaced in a proximal direction to set the fixed dose of the second medicament, and wherein, when the dose setter is detached from the fixed dose setting mechanism, the dose setter can be rotated to set the user settable dose of the first medicament.

6. The drug delivery device of claim 1, wherein the dose setter is configured to set the fixed dose of the second medicament before the user settable dose of the first medicament.

7. The drug delivery device of claim 1, wherein the dose setter detaches from the fixed dose setting mechanism after the fixed dose of the second medicament is set.

8. The drug delivery device of claim 1, wherein, after (i) the user settable dose of the first medicament and the fixed dose of the second medicament are set and (ii) the user settable dose of the first medicament is subsequently delivered, the dose setter re-attaches to the fixed dose setting mechanism via the connecting feature such that axial displacement of the dose setter in the distal direction causes the fixed dose of the second medicament to be delivered.

9. The drug delivery device of claim 1 wherein the dose setter is part of the rotationally driven variable dose setting mechanism.

10. The drug delivery device of claim 1, wherein the dose setter is configured to set the fixed dose of the second medicament before the user settable dose of the first medicament, and wherein both the fixed dose of the second medicament and the user settable dose of the first medicament are set by rotating the dose setter.

11. The drug delivery device of claim 1, wherein, after the fixed dose of the second medicament is set, the rotationally driven variable dose setting mechanism detaches from the fixed dose setting mechanism.

12. The drug delivery device of claim 1, wherein, after the fixed dose of the second medicament and the user settable dose of the first medicament are set, and the drug delivery device is subsequently activated, the user settable dose of the first medicament is delivered before the fixed dose of the second medicament.

13. The drug delivery device of claim 12, wherein, after the user settable dose of the first medicament is delivered, the rotationally driven variable dose setting mechanism re-attaches to the fixed dose setting mechanism via the connecting feature such that axial displacement of the connecting feature in a distal direction causes the fixed dose of the second medicament to be delivered.
